(19) United States
(12) Patent Application Publication
(10) Pub. No.: US 2023/0410964 A1
(43) Pub. Date: Dec. 21, 2023

(54) INFORMATION SYSTEM, INFORMATION PROCESSING METHOD, AND STORAGE MEDIUM

(71) Applicant: CANON KABUSHIKI KAISHA, Tokyo (JP)

(72) Inventors: Genki CHO, Kanagawa (JP); Satoshi ISHIZU, Tokyo (JP)

(21) Appl. No.: 18/332,015

(22) Filed: Jun. 9, 2023

(30) Foreign Application Priority Data
Jun. 21, 2022 (JP) 2022-099802

(51) Int. Cl. G16H 10/60; H04N 23/661; H04N 23/63

(52) U.S. Cl. CPC G16H 10/60; H04N 23/661; H04N 23/634; G16H 30/40

(57) ABSTRACT

A first obtainment unit of a control apparatus obtains first information pertaining to a first patient from an electronic medical record terminal in response to a patient for display of an electronic medical record changing to the first patient in the electronic medical record terminal. The control apparatus is capable of communicating with the electronic medical record terminal. An association unit associates the first information with a first image captured between when the patient for display is changed to the first patient and when the patient for display is changed to another patient. The first image is captured by a specific image capturing apparatus capable of communicating with the control apparatus.

### BACKGROUND OF THE INVENTION

### Field of the Invention

The present invention relates to an information system, an information processing method, and a storage medium.

### Description of the Related Art

Image capturing apparatuses, imaging systems, or the like that capture images of an affected part of a subject (a patient), record progress, assist in diagnoses, and so on are known. Japanese Patent Laid-Open No. 2019-171095 discloses an image capturing and processing system that adds information based on the state of an affected part of a subject (e.g., a patient's chief complaint, a photographer's findings, and so on) to image data generated by image capturing processing.

In medical settings and the like, some work is required to manage information pertaining to a patient (such as the patient's name) in association with images obtained by shooting images of the patient. With conventional techniques, including Japanese Patent Laid-Open No. 2019-171095, it has been necessary for a doctor, a photographer, or the like to identify a patient and associate information pertaining to the patient with that patient, and thus the work involved in this association has been relatively labor-intensive.

### SUMMARY OF THE INVENTION

Having been achieved in light of such circumstances, the present invention provides a technique that reduces a workload of associating information pertaining to a patient with an image obtained by capturing an image of the patient.

According to a first aspect of the present invention, there is provided an information system comprising: a control apparatus including a first obtainment unit configured to obtain first information pertaining to a first patient from an electronic medical record terminal in response to a patient for display of an electronic medical record changing to the first patient in the electronic medical record terminal, the control apparatus being capable of communicating with the electronic medical record terminal; and an association unit configured to associate the first information with a first image captured between when the patient for display is changed to the first patient and when the patient for display is changed to another patient, the first image being captured by a specific image capturing apparatus capable of communicating with the control apparatus.

According to a second aspect of the present invention, there is provided an information processing method comprising: obtaining first information pertaining to a first patient from an electronic medical record terminal by a first obtainment unit of a control apparatus capable of communicating with the electronic medical record terminal, the obtaining being performed in response to a patient for display of an electronic medical record changing to the first patient in the electronic medical record terminal; and associating, by an association unit, the first information with a first image captured between when the patient for display is changed to the first patient and when the patient for display is changed to another patient, the first image being captured by a specific image capturing apparatus capable of communicating with the control apparatus.

According to a third aspect of the present invention, there is provided a non-transitory computer-readable storage medium which stores a program for causing a computer system, which includes a control apparatus capable of communicating with an electronic medical record terminal, to execute an information processing method comprising: obtaining first information pertaining to a first patient from the electronic medical record terminal, the obtaining being performed in response to a patient for display of an electronic medical record changing to the first patient in the electronic medical record terminal; and associating the first information with a first image captured between when the patient for display is changed to the first patient and when the patient for display is changed to another patient, the first image being captured by a specific image capturing apparatus capable of communicating with the control apparatus.

Further features of the present invention will become apparent from the following description of exemplary embodiments with reference to the attached drawings.

### BRIEF DESCRIPTION OF THE DRAWINGS

FIG. 1 is a diagram illustrating the configuration of a medical imaging system.

FIG. 2 is a block diagram illustrating a digital still camera operating as the image capturing apparatus 101.

FIG. 3 is a block diagram illustrating a computer that operates as an imaging system control apparatus 106.

FIG. 4 is a flowchart illustrating imaging processing performed by an imaging system.

FIG. 5 is a diagram illustrating an image capturing apparatus 101 from the rear.

FIG. 6 is a diagram illustrating an example of a warning message in the imaging processing.

FIG. 7 is a diagram illustrating a display state of a display unit 204 of the image capturing apparatus 101 when medical record information has been deleted.

FIG. 8 is a diagram illustrating the configuration of an imaging system for a transport system.

FIG. 9 is a flowchart illustrating the flow of processing for recording a problem state and the like of an apparatus using the imaging system for the transport system (recording processing).

FIG. 10 is a diagram illustrating an example of apparatus information displayed in the image capturing apparatus 101.

FIG. 11 is a flowchart illustrating imaging processing in an imaging system that may include a plurality of image capturing apparatuses 101.

FIG. 12 is a flowchart illustrating recording processing in a transport system that may include a plurality of image capturing apparatuses 101.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate. Furthermore, in the attached drawings, the same reference numerals are given to the same or similar configurations, and redundant description thereof is omitted.

### First Embodiment

The present embodiment will describe an imaging system used to capture an affected part of a patient and assist in a diagnosis in a medical setting. For example, the imaging system according to the present embodiment is used by a doctor to capture an image of an affected part and store photographer information, patient information, and the like with image data of the affected part as metadata.

Although an image capturing apparatus (a digital camera) capable of capturing visible light is assumed to be used in the present embodiment, the type of the image capturing apparatus is not particularly limited. A multi spectral camera capable of capturing different wavelength regions according to the medical purpose and imaging purpose, a polarization camera having a polarizer that transmits light which oscillates in a certain direction, and an infrared camera capable of capturing infrared to near-infrared regions can be given as other examples of the image capturing apparatus. A three-dimensional shape measurement camera capable of capturing three-dimensional shapes, a microscope-type camera, a dermoscope-type camera, a camera having a plurality of such image capturing functions, and the like can be given as further examples.

System Configuration

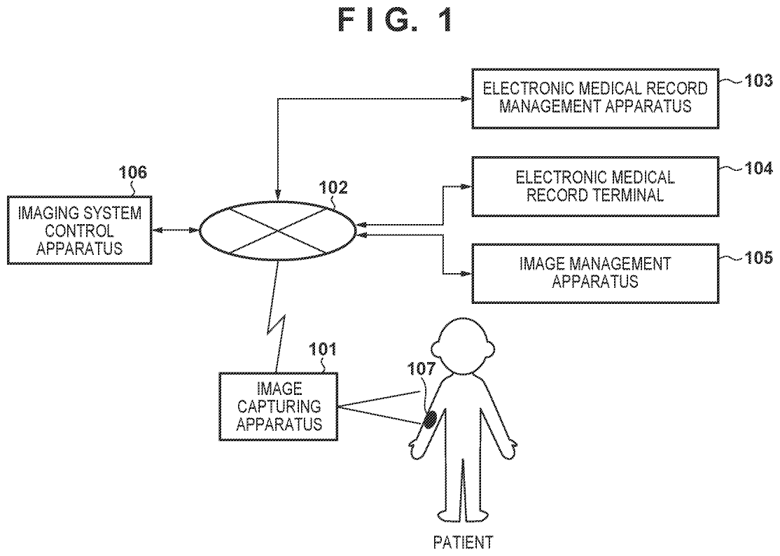

FIG. 1 is a diagram illustrating the configuration of a medical imaging system. The imaging system illustrated in FIG. 1 functions as an information system that performs processing (information processing) such as associating information pertaining to a patient with a captured image. In the imaging system, an image capturing apparatus 101 generates image data by capturing an image of a subject (e.g., an affected part 107 of a patient). The following descriptions assume that the image data captured by the image capturing apparatus 101 is visible light image data. Like a commercially-available digital camera, the image capturing apparatus 101 is a portable, compact image capturing apparatus, and is provided with a display device through which captured images can be confirmed and an interface, such as buttons and a touch panel, for making various types of operations.

A network 102 is a communication network that enables communication between apparatuses in the imaging system. Although the term “apparatus” is not included in the term “electronic medical record terminal 104”, the term “apparatus” used in the present specification as a generic term is assumed to include the electronic medical record terminal 104 as long as doing so is not technically inconsistent.

For example, when the power is turned on by a photographer, the image capturing apparatus 101 can connect to the network 102 and communicate with other apparatuses in the imaging system over the network 102. The network 102 performs wireless data transmission, wired data transmission, various types of control, and the like. Although the communication protocol used for communication over the network 102 is not particularly limited, Hypertext Transfer Protocol (HTTP) and File Transfer Protocol (FTP) can be given as examples thereof. Picture Transfer Protocol (PTP), Universal Serial Bus (USB), and Local Area Network (LAN) can be given as other examples. Health Level Seven (HL7) and Digital Imaging and Communications in Medicine (DICOM) can be given as further examples. Any communication protocol may be used as long as image data, text, control data, and the like can be communicated.

In the present embodiment, each apparatus, including the image capturing apparatus 101, is assumed to maintain a state in which communication can be continued until the power is turned off. Although a method for connection and communication between the image capturing apparatus 101 and the network 102 has been described in detail above, the same applies to various other apparatuses such as an electronic medical record management apparatus 103, the electronic medical record terminal 104, an image management apparatus 105, an imaging system control apparatus 106, and the like. Although not illustrated in FIG. 1, a lighting apparatus constituted by a halogen light, a light-emitting diode light, or the like may be attached for capturing images as necessary.

The electronic medical record management apparatus 103 stores patient information (an ID, name, medical department, age, sex, medical history, lesion, and so on) and doctor information (an ID, name, affiliation information such as medical department, and so on). The electronic medical record management apparatus 103 can verify and authenticate login/logoff information of a doctor entered in the electronic medical record terminal 104, provide doctor information to the electronic medical record terminal 104, provide patient information to the electronic medical record terminal 104 in the form of an electronic medical record, and the like. The electronic medical record management apparatus 103 can also store data updated by the electronic medical record terminal 104. The electronic medical record management apparatus 103 can be implemented as a personal computer (PC), a server in a hospital, or a cloud server. The electronic medical record terminal 104 can access the electronic medical record management apparatus 103 via the network 102 when necessary.

When a patient is examined by a doctor, the electronic medical record terminal 104 provides an electronic medical record function by downloading information of the doctor who is logged in and information of the patient being examined from the electronic medical record management apparatus 103. This makes it possible to display, edit, and update the electronic medical record.

The image management apparatus 105 can store, update, or delete image data, as well as photographer information and patient information associated with the image data. The image management apparatus 105 also includes a data search function and the like as necessary.

The imaging system control apparatus 106 obtains the above-described photographer information, patient information, and the like from the electronic medical record terminal 104, transmits the obtained information to the image capturing apparatus 101, and the like. When capturing an image, the image capturing apparatus 101 can temporarily store the photographer information, patient information, and the like transmitted from the imaging system control apparatus 106 in association with the image data. Thereafter, the imaging system control apparatus 106 detects various types of events such as image capturing events of the image capturing apparatus 101, and receives various types of data associated with the image data that has been captured. The imaging system control apparatus 106 transmits various types of data, such as received image data and the like, to the electronic medical record terminal 104 and the image management apparatus 105. The processing executed by the imaging system control apparatus 106 and the image capturing apparatus 101 will be described in detail later.

Additionally, the imaging system control apparatus 106 may perform image processing (e.g., thumbnail extraction, data compression, pathology estimation, and so on) on the image data received from the image capturing apparatus 101 according to the purpose of the examination. The imaging system control apparatus 106 can also display the result of the image processing in a display unit 304 (described later), transmit the result of the image processing to the electronic medical record terminal 104 over the network 102 and cause the electronic medical record terminal 104 to display the result, and so on. The imaging system control apparatus 106 may also transmit the result of the image processing to the image capturing apparatus 101 and cause the image capturing apparatus 101 to display the result in a display unit 204. Additionally, the imaging system control apparatus 106 may transmit the data for which image processing is complete to the image management apparatus 105. Configuring the imaging system control apparatus 106 in this manner makes it possible for a user of the imaging system to confirm image data when capturing images, confirm the results of image processing on image data from images captured of the patient during medical procedures, and the like.

In the present embodiment, the image capturing apparatus 101, the electronic medical record management apparatus 103, the electronic medical record terminal 104, the image management apparatus 105, and the imaging system control apparatus 106 are described as separate apparatuses, but the present embodiment is not limited to such a configuration. For example, a single apparatus may realize functions of at least two of the apparatuses, by, for example, constructing at least some of the electronic medical record terminal 104, the electronic medical record management apparatus 103, the imaging system control apparatus 106, and the image management apparatus 105 within the same housing. Doing so makes it possible to configure a system having a cost that is appropriate relative to the load and capacity required by the system.

Configuration of Image Capturing Apparatus 101

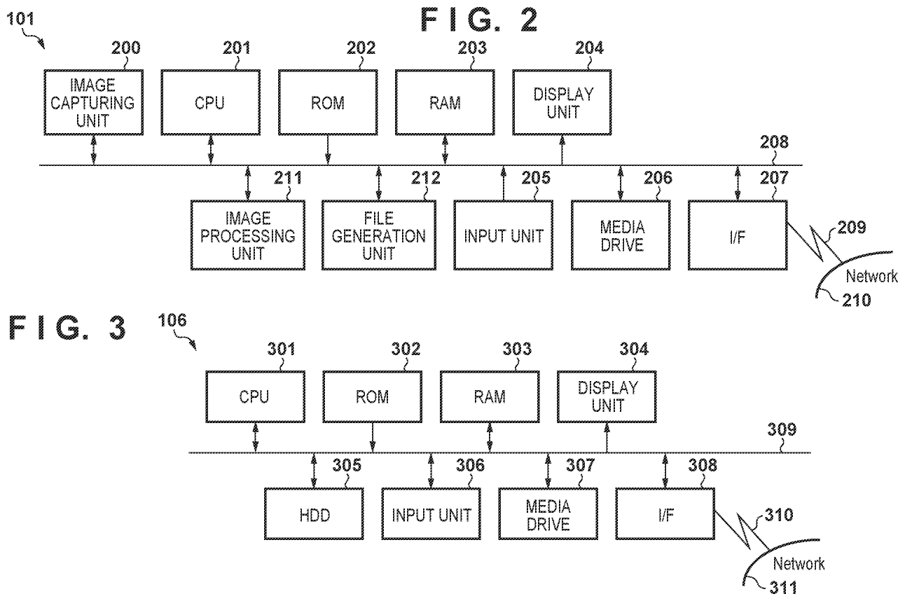

A digital still camera that operates as the image capturing apparatus 101 will be described next with reference to FIG. 2. FIG. 2 is a block diagram illustrating a digital still camera operating as the image capturing apparatus 101. The digital still camera implements the image capturing processing described below by executing a predetermined control program, and functions as the image capturing apparatus 101.

200 indicates an image capturing unit that reads an optical image using a solid-state image sensor and generates electrical image data through analog-digital conversion.

201 indicates a CPU that controls the image capturing apparatus 101 as a whole.

202 indicates a Read-Only Memory (ROM) that stores an operation processing sequence of the CPU 201 (e.g., programs for processing for turning on the power of the image capturing apparatus 101, basic input/output processing, and so on).

203 indicates a Random Access Memory (RAM) that functions as the main memory (a main temporary storage unit) for the CPU 201. The main memory of the CPU 201 may be a storage unit that is different from the RAM. For example, the main memory may be constituted by a non-volatile memory device such as a Read-Only Memory (ROM) or an Electrically Erasable Programmable Read-Only Memory (EEPROM). As another example, the main memory may be constituted by a file region, a virtual storage region, or the like in an external storage apparatus (not shown) connected over a network or the like. Various types of programs, including control programs for realizing the processing described later, are loaded from the ROM 202 or the like into the RAM 203 and executed by the CPU 201. The RAM 203 also provides a work area when the CPU 201 executes various processing.

204 indicates a display unit that performs various displays under the control of the CPU 201. For example, the display unit 204 displays data stored in the ROM 202, the RAM 203, a media drive 206, or the like. Additionally, the display unit 204 may display live view images captured by the image capturing unit 200, captured images captured by the image capturing unit 200 in response to an image capture instruction being input when a release button is operated, various types of settings screens, and the like.

205 indicates an input unit including buttons and the like for performing various types of operations. “Buttons or the like” refers to, for example, a release button located on top of the image capturing apparatus 101, a directional key and a setting key located on the rear, and the like. The input unit 205 also includes a touch panel or the like provided on the display unit 204. The user (photographer) can input various types of instructions to the image capturing apparatus 101 by operating the input unit 205.

206 indicates a media drive, which includes a mounting unit for mounting a removable storage medium. The media drive 206 enables data to be stored in the storage medium, stored data to be read out, and the like.

207 indicates a network interface (a communication unit). The image capturing apparatus 101 sends and receives data to and from a server computer, a personal computer, or the like through the network interface 207.

208 indicates a system bus (including an address bus, a data bus, and a control bus) for connecting various units to one another.

209 indicates a wired or wireless communication line.

210 indicates a computer network. The image capturing apparatus 101 is connected to the computer network 210 by the communication line 209.

211 indicates an image processing unit. The CPU 201 temporarily stores the image data generated by the image capturing unit 200 and attribute information thereof in the RAM 203. Then, if necessary, the image processing unit 211 performs a series of image processing to obtain image data that meets the imaging purpose, such as data including, for example, the vision characteristics of humans, three-dimensional data, or both. If the processing load is high, the image processing unit 211 may divide the processing for the data and share the processing with the imaging system control apparatus 106.

212 indicates a file generation unit. For example, the file generation unit 212 generates an image file by converting image data into a general-purpose still image format.

Configuration of Imaging System Control Apparatus 106

A computer that operates as the imaging system control apparatus 106 will be described with reference to FIG. 3. FIG. 3 is a block diagram illustrating a computer that operates as the imaging system control apparatus 106. By executing a predetermined control program, the computer implements the processing described below and functions as the imaging system control apparatus 106. Note that the computer may function by being provided in a server on a local network, the cloud, or the like.

301 indicates a CPU that controls the imaging system control apparatus 106 as a whole.

302 indicates a ROM that stores an operation processing sequence of the CPU 301 (e.g., programs for startup processing of the imaging system control apparatus 106, basic input/output processing, and the like).

303 indicates a RAM, which functions as main memory for the CPU 301. Various types of programs, including control programs for realizing the processing described later, are loaded from an HDD 305 (a hard disk drive) or the like into the RAM 303 and executed by the CPU 301. The RAM 303 also provides a work area when the CPU 301 executes various processing.

304 indicates a display unit that performs various displays under the control of the CPU 301.

305 indicates a hard disk drive (HDD), which is used to store and load application programs, data, libraries, and the like.

306 indicates an input unit including a pointing device, a keyboard, and the like.

307 indicates a media drive, which includes a mounting unit for mounting a removable storage medium. The imaging system control apparatus 106 can read out data captured by the image capturing apparatus 101 and stored in the storage medium through the media drive 307.

308 indicates a network interface (a communication unit). The imaging system control apparatus 106 sends and receives data to and from external apparatuses using the network interface 308. In the present embodiment, the imaging system control apparatus 106 is connected to the image capturing apparatus 101 through the network interface 308. The CPU 301 then sends and receives various types of data to and from the image capturing apparatus 101, obtains captured images from the image capturing apparatus 101, and records the images in the HDD 305.

309 indicates a system bus (including an address bus, a data bus, and a control bus) for connecting various units to one another.

310 indicates a wireless or wired communication line.

311 indicates a computer network. The imaging system control apparatus 106 is connected to the computer network 311 by the wireless or wired communication line 310.

User Interface of Image Capturing Apparatus 101

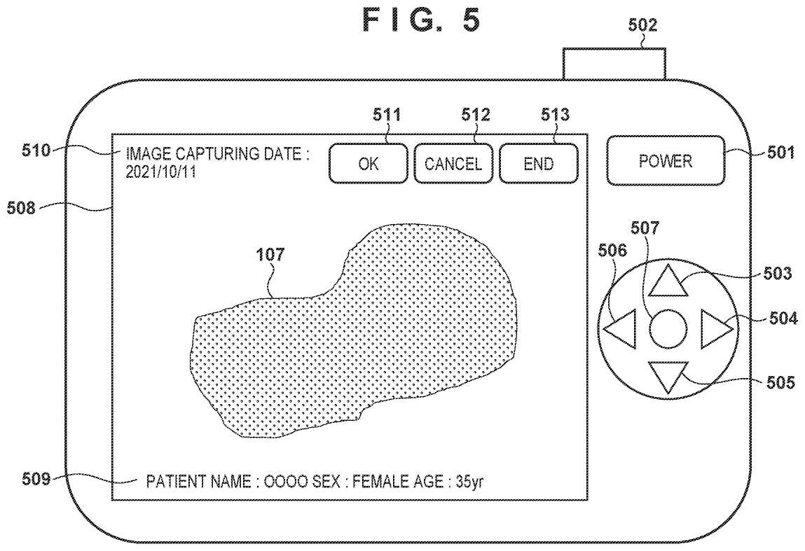

FIG. 5 is a diagram illustrating the image capturing apparatus 101 from the rear. The various buttons and the user interface of the image capturing apparatus 101 will be described in detail with reference to FIG. 5. Aside from the power button 501, the operations of all the buttons will be described assuming a state in which the image capturing apparatus 101 is powered on.

501 indicates a power button that switches the power on and off. When the photographer presses the power button 501 while the power of the image capturing apparatus 101 is not on, the CPU 201 determines that an instruction to turn the power on has been received from the photographer, and turns the power on. When the photographer presses the power button while the power is on, the CPU 201 determines that an instruction to turn the power off has been received from the photographer, and turns the power off.

502 indicates a release button. When the photographer presses the release button 502, the CPU 201 determines that an instruction to capture a still image has been made. 503 indicates an upward direction button, 504 indicates a rightward direction button, 505 indicates a downward direction button, 506 indicates a leftward direction button, and 507 indicates a confirmation button. These buttons implement the functions of the input unit 205.

When a button indicated by the numbers 503 to 506 is pressed by the photographer, the CPU 201 determines that an instruction to switch the selection target has been received from the photographer, and switches the selection target in a display 508 (described later). When the photographer presses the confirmation button 507, the CPU 301 determines that the photographer has made a confirmation instruction, holds the selected information in the RAM 303, and switches the state of the image capturing apparatus 101.

508 indicates a display, which implements the functions of the display unit 204. In the example illustrated in FIG. 5, the display 508 displays an image of the patient's affected part 107. The display 508 may have touch panel functionality along with display functionality, and may therefore implement the functions of the input unit 205. In this case, when the photographer touches a desired point on the screen with a finger, the CPU 201 determines that an input instruction has been made from the photographer, determines the details of the operation from the touched position, and performs various types of processing, such as updating the display, updating settings, and the like. For example, a configuration may be employed in which an OK button 511, a cancel button 512, an end button 513, and the like are arranged in the screen, and the photographer's instructions are input through these buttons.

509 is an example of a patient information display. In the example in FIG. 5, the name, sex, and age of the patient are displayed. By displaying the patient information superimposed on a subject image when capturing an image in this manner, the doctor can ensure that the patient information to be associated with the patient being captured is not mistaken. In the present embodiment, the patient information is received along with doctor information from the imaging system control apparatus 106. 510 indicates date/time information on the time the image is captured. Note that the display 508 may further display the doctor information as the photographer information, and may switch the display information based on an operation status, priority, and the like in the hospital.

Flow of Imaging Processing

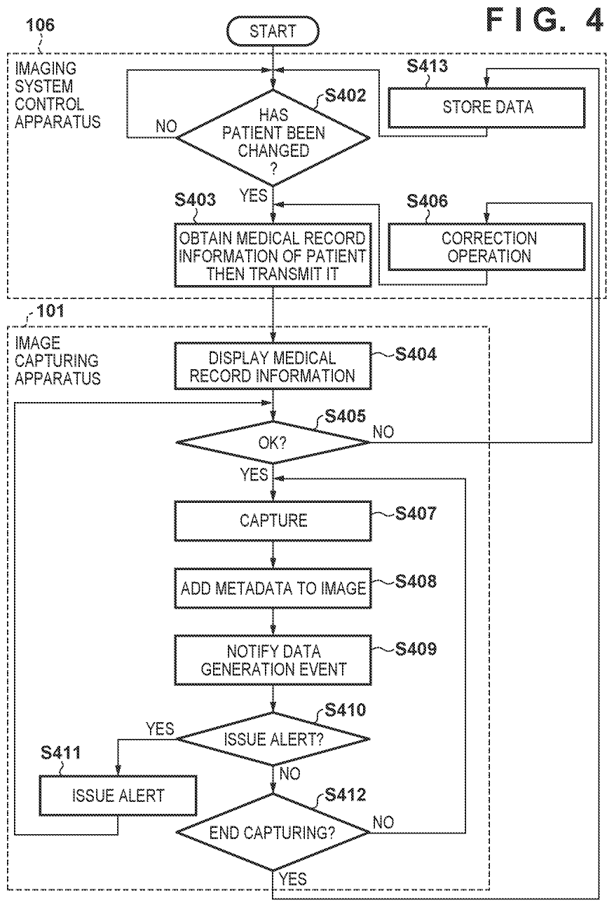

FIG. 4 is a flowchart illustrating imaging processing performed by the imaging system. The imaging processing is performed, for example, to record the state of a patient's injury or illness. Although the following descriptions assume that the photographer is a doctor, the photographer may be a technician, an assistant, a nurse, or the like designated by the doctor. It is assumed that the doctor has logged in to the electronic medical record terminal 104 prior to the imaging processing being performed. Once the doctor has logged into the electronic medical record terminal 104, the electronic medical record terminal 104 obtains the patient information (ID, name, medical department, age, sex, medical history, lesion, and the like) and the doctor information (ID, name, affiliation information such as a medical department, and the like) from the electronic medical record management apparatus 103. The electronic medical record terminal 104 can then display an electronic medical record based on the information obtained from the electronic medical record management apparatus 103.

The imaging processing includes processing performed by the image capturing apparatus 101 and processing performed by the imaging system control apparatus 106. Unless otherwise specified, the processing by the image capturing apparatus 101 is realized by the CPU 201 of the image capturing apparatus 101 executing a control program. Additionally, unless otherwise specified, the processing by the imaging system control apparatus 106 is realized by the CPU 301 of the imaging system control apparatus 106 executing a control program.

Note that dividing the processing among the apparatuses in the imaging system is not limited to the division described hereinafter and illustrated in FIG. 4. For example, if technically feasible, the processing described hereinafter as being executed by the image capturing apparatus 101 may be executed by the imaging system control apparatus 106 or another apparatus, such as the electronic medical record terminal 104. A plurality of apparatuses may also execute the same processing. For example, if technically feasible, the processing described hereinafter as being executed by the image capturing apparatus 101 may be executed by the image capturing apparatus 101 and the imaging system control apparatus 106, respectively.

In step S402, the CPU 301 of the imaging system control apparatus 106 determines whether a patient whose electronic medical record is displayed in the electronic medical record terminal 104 (a patient for whom the electronic medical record is to be displayed) has been changed. The present embodiment assumes that the doctor can select the patient for whom the electronic medical record is to be displayed by operating the electronic medical record terminal 104. Accordingly, a change in the patient for display occurs in response to the patient for display being selected by the doctor. The CPU 301 repeats the determination in step S402 until the patient for display is changed, and the processing moves to step S403 in response to the patient for display being changed.

Note that the specific configuration for realizing the determination in step S402 is not particularly limited, and any appropriate configuration can be employed in accordance with the conventions of who is deploying the imaging system, or the policies of where the imaging system is being deployed, the protocol for connecting the apparatuses in the imaging system to each other, and the like. For example, a configuration may be employed in which the electronic medical record terminal 104 notifies the imaging system control apparatus 106 of a change in the patient for display over the network 102. In this case, the CPU 301 of the imaging system control apparatus 106 can determine that the patient for display has changed in response to receiving the notification from the electronic medical record terminal 104. As another example, a configuration may be employed in which the CPU 301 of the imaging system control apparatus 106 determines whether the patient for display has changed by monitoring and detecting changes in the patient for display in the electronic medical record terminal 104. In this case, for example, the CPU 301 of the imaging system control apparatus 106 may monitor and detect changes in the patient for display by monitoring the screen of the electronic medical record terminal 104.

In step S403, the CPU 301 obtains information pertaining to the patient for display (first information) after the change (a patient for which an electronic medical record is newly displayed) from the electronic medical record terminal 104, and transmits the first information to the image capturing apparatus 101. Although the information pertaining to the patient for display obtained here is not particularly limited, the following descriptions will assume that the information is information recorded in the electronic medical record of the patient for display (medical record information). Examples of the medical record information include the doctor's ID, the doctor's name, the medical department, the patient's ID, the patient's name, age, sex, medical history, image capturing history, affected part, lesion, and the like. The following descriptions will assume that the medical record information obtained here includes the doctor's ID, the patient's ID, and the patient's name, age, and sex.

The patient for which an image is captured by the imaging system is likely to be a patient for whom the electronic medical record is displayed in the electronic medical record terminal 104 when the image is captured (the patient for whom the electronic medical record is to be displayed when the image is captured). In light of this point, the imaging processing illustrated in FIG. 4 is configured such that the CPU 301 of the imaging system control apparatus 106 obtains information pertaining to the patient for display after the change (a first patient) from the electronic medical record terminal 104 in response to the patient for display having been changed. This makes it possible to easily associate the information pertaining to the patient for whom the image has been captured with the captured image, which lightens the burden of the association task.

In step S404, the CPU 201 of the image capturing apparatus 101 displays at least some of the medical record information received from the imaging system control apparatus 106 in the display unit 204. In the example illustrated in FIG. 5, as described earlier, the name, sex, and age of the patient are displayed in the display 508 of the display unit 204 (see the patient information display 509). The user (a photographer such as a doctor) confirms that the patient indicated by the medical record information displayed in the display unit 204 matches the patient for whom an image is being captured. If the patient indicated by the medical record information matches the patient for whom an image is being captured, the user selects the OK button 511. If the patient indicated by the medical record information does not match the patient for whom an image is being captured, the user selects the cancel button 512. A discrepancy between the patient indicated by the medical record information and the patient for whom an image is being captured occurs when, for example, the doctor has forgotten to switch the patient for whom the electronic medical record is to be displayed in the electronic medical record terminal 104, has failed to transmit the correct medical record information to the image capturing apparatus 101 due to a communication error in the imaging system, or the like.

In step S405, the CPU 201 determines the result of the selection of the button by the user. If the OK button 511 has been selected, the sequence moves to step S407, whereas if the cancel button 512 has been selected, the sequence moves to step S406.

In step S406, the CPU 301 of the imaging system control apparatus 106 displays a user interface for correcting the medical record information in the display unit 304. The user performs the necessary correction task through the user interface displayed in the display unit 304. The information in the electronic medical record is updated as a result. The sequence then moves to step S403, where the CPU 301 of the imaging system control apparatus 106 transmits the updated medical record information to the image capturing apparatus 101. In this manner, providing a configuration that enables the user to confirm the medical record information (patient information and the like), correct (update) the information as necessary, and so on makes it possible to prevent erroneous associations from being made between the image data of the patient for whom an image is being captured and the medical record information of the patient.

Although a configuration in which the user corrects the medical record information through the user interface displayed in the display unit 304 of the imaging system control apparatus 106 is described here, a configuration may be employed in which the user can perform the same work in the display unit of the electronic medical record terminal 104 or the display unit 204 of the image capturing apparatus 101.

In step S407, the CPU 201 of the image capturing apparatus 101 performs image capturing processing in response to the user pressing the release button 502. The image data obtained from capturing the image is temporarily stored in the RAM 203, the ROM 202, the media drive 206, or the like.

In step S408, the CPU 201 adds, as metadata, the medical record information transmitted from the imaging system control apparatus 106 in step S403 to the image data captured in step S407. The image data and the medical record information are associated as a result. Although the method for this association is not particularly limited, for example, the CPU 201 can write medical record information into a metadata region or the like of the image data. Alternatively, the CPU 201 may control the file generation unit 212 to generate a metadata file containing the medical record information, and associate the image data file with the metadata file.

Here, the CPU 201 of the image capturing apparatus 101 is described as associating the medical record information (metadata) with the captured image. However, instead of the CPU 201 of the image capturing apparatus 101, the CPU 301 of the imaging system control apparatus 106 may associate the medical record information (metadata) with the captured image. In this case, in step S403, the CPU 301 of the imaging system control apparatus 106 obtains the medical record information from the electronic medical record terminal 104, but the obtained medical record information need not be transmitted to the image capturing apparatus 101. The CPU 301 of the imaging system control apparatus 106 then associates the medical record information with a downloaded image in accordance with a data generation event of step S409 (described later).

In step S409, the CPU 201 notifies the imaging system control apparatus 106 of a data generation event. The “data generation event” is an event indicating that image data to which metadata has been added has been generated by the image capturing apparatus 101. Accordingly, upon receiving the notification of the data generation event, the CPU 301 of the imaging system control apparatus 106 can recognize that new image data has been generated by the image capturing apparatus 101, and can receive (download) that image data.

Note that the timing at which the CPU 301 of the imaging system control apparatus 106 downloads the image data from the image capturing apparatus 101 is not particularly limited. For example, the CPU 301 of the imaging system control apparatus 106 can download the image data in response to receiving the notification of the data generation event. In this case, the image data is downloaded each time the image capturing apparatus 101 captures an image. Alternatively, the CPU 301 of the imaging system control apparatus 106 may later download, in a batch, the image data corresponding to a plurality of captured images stored in a storage region of the image capturing apparatus 101 (such as the ROM 202, the media drive 206, or the like). Although the timing of the batch download is not particularly limited, for example, the timing at which the image capturing of a particular patient is thought to have been completed (e.g., the timing at which the patient for whom the electronic medical record is to be displayed has been changed to another patient, the timing at which the doctor has started entering information into a specific region, such as a diagnostic result, in the electronic medical record, or the like) is conceivable. This configuration makes it possible to suppress the occurrence of situations where the download time is delayed for each capture in medical departments where a high number of images are captured per patient, hospitals for which the bandwidth of the network 102 is limited, and so on, which eliminates stress on the part of the photographer.

The CPU 201 of the image capturing apparatus 101 may display a user interface (a first user interface) that includes the captured image (a first image) and a cancellation option in response to the image being captured in step S407. The cancellation option is an option that can be selected by the user, such as the cancel button 512 illustrated in FIG. 5, for example. When the cancellation option is selected in the user interface, the CPU 201 of the image capturing apparatus 101 cancels the aforementioned data generation event (a first notification) for the imaging system control apparatus 106. If a predetermined length of time (a first time) has passed without the cancellation option being selected in the user interface, the CPU 201 of the image capturing apparatus 101 makes the aforementioned data generation event (the first notification) for the imaging system control apparatus 106. This makes it possible to suppress a situation where an image that the user considers unnecessary is downloaded by the imaging system control apparatus 106.

In step S410, the CPU 201 of the image capturing apparatus 101 determines whether to alert the user to confirm the medical record information (a second notification or a third notification). If it is determined that the user is to be alerted, the sequence moves to step S411, and if not, the sequence moves to step S412.

The determination here is made based on the possibility that the doctor has forgotten to switch the patient for whom the electronic medical record is to be displayed. For example, when image capturing using the same medical record information continues for at least a predetermined length of time (i.e., after the patient for display has been changed to the current patient (the first patient) and a predetermined length of time (a second time) has passed before being changed to another patient), it is likely that the doctor has forgotten to switch the patient for whom the electronic medical record is to be displayed. Accordingly, the CPU 201 determines to issue the alert. As another example, when a predetermined number of images have been captured using the same medical record information (i.e., when a predetermined number of images have been captured after the patient for display has been changed to the current patient (the first patient) but before being changed to another patient), it is likely that the doctor has forgotten to switch the patient for whom the electronic medical record is to be displayed. Accordingly, the CPU 201 determines to issue the alert.

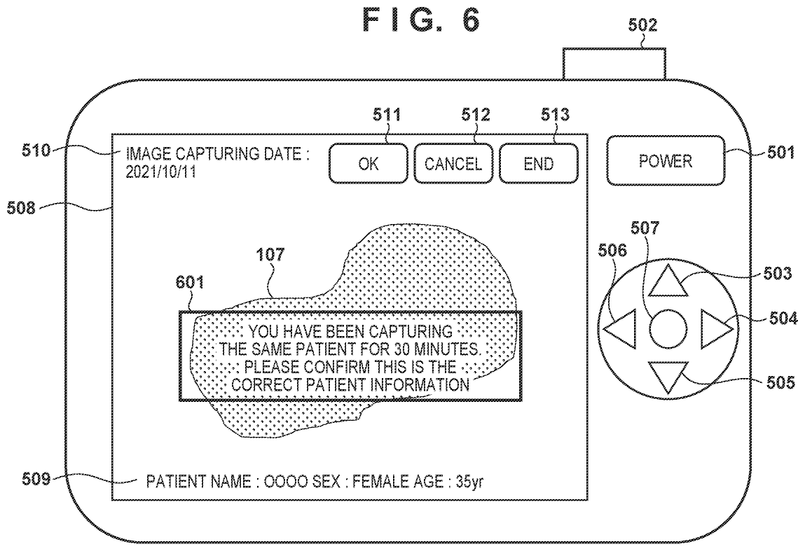

In step S411, the CPU 201 alerts the user to confirm the medical record information. Although the alert method is not particularly limited, for example, the CPU 201 can display a warning message 601 such as that illustrated in FIG. 6 in the display 508 when images have been captured using the same medical record information for at least 30 minutes. The sequence then moves to step S405. Like the aforementioned process of step S405, the CPU 201 determines whether the user has selected the OK button 511 or the cancel button 512, and the sequence moves to step S405 or step S406 according to the result of the determination. Making an alert in this manner makes it possible to suppress inconsistencies in the metadata associated with the image data that has been captured (for example, associating the medical record information of the patient from before the change with the image data despite the patient being captured have been changed).

The foregoing descriptions assume that the CPU 201 of the image capturing apparatus 101 makes the determination in step S410 and alerts the user in step S411. However, a configuration may be employed in which the CPU 301 of the imaging system control apparatus 106 makes the determination in step S410 and alerts the user in step S411. Additionally, the CPU 301 of the imaging system control apparatus 106 may notify the image capturing apparatus 101, the electronic medical record terminal 104, or the like of the determination made in step S410. In this case, the image capturing apparatus 101, the electronic medical record terminal 104, or the like can issue the alert in response to the notification from the imaging system control apparatus 106. Additionally, the alert may be issued simultaneously by a plurality of apparatuses, such as the image capturing apparatus 101, the imaging system control apparatus 106, and the electronic medical record terminal 104. Such a configuration makes it possible to suppress inconsistencies in the metadata associated with the image data, regardless of the gaze and behavior of the doctor, the status of the examination, or the like.

In step S412, the CPU 201 of the image capturing apparatus 101 determines whether to end the capturing. The user selects the end button 513 when all the necessary affected parts have been captured and the user wishes to end the capturing. When the end button 513 is selected, the CPU 201 determines to end the capturing. When it is determined that shooting is not to be ended, the sequence returns to step S407. When it is determined that the shooting is to be ended, the CPU 201 deletes the medical record information held by the image capturing apparatus 101 (the medical record information transmitted from the imaging system control apparatus 106 in step S403). The sequence then moves to step S413.

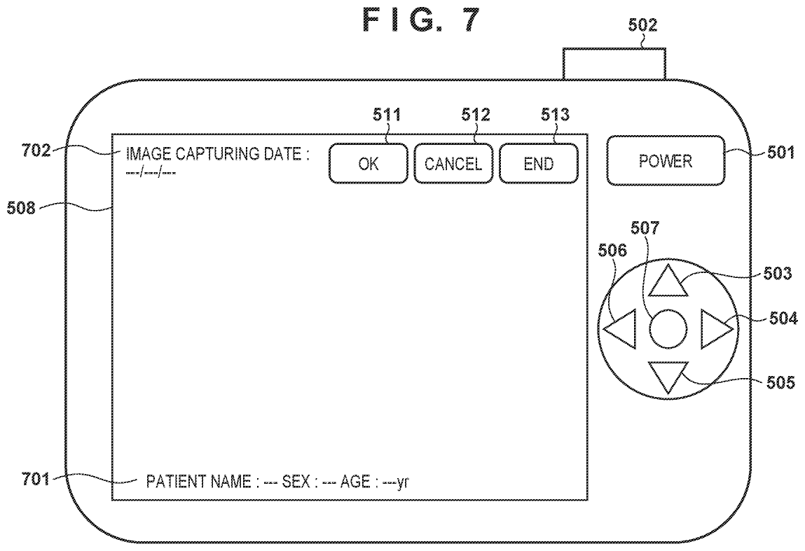

FIG. 7 is a diagram illustrating a display state of the display unit 204 of the image capturing apparatus 101 when the medical record information has been deleted. As illustrated in FIG. 7, a patient information display 701 does not include the medical record information. Additionally, an image capture date/time 702 does not include date/time information. In this manner, deleting the metadata (the medical record information) in the image capturing apparatus 101 each time the image capturing for a single patient ends makes it possible to suppress situations where incorrect metadata is associated with the image data. Meanwhile, the user can recognize that the shooting has ended upon seeing the display state illustrated in FIG. 7.

Note that the image capturing apparatus 101 may be configured so as not to capture an image before the medical record information is received, even if the release button 502 is pressed. This makes it possible to suppress the generation of image data in which the photographer and the subject are not clear. Additionally, a configuration may be employed that enables the user to erase the storage of the image data, metadata, and the like after the user has finished capturing the affected part. For example, in response to the user selecting the cancel button 512 after the affected part has been captured, the image capturing apparatus 101 deletes the image data temporarily stored therein along with the corresponding metadata. Additionally, the image capturing apparatus 101 requests the imaging system control apparatus 106 to delete the image data and the corresponding metadata that the imaging system control apparatus 106 has stored up to that point. Doing so makes it possible to reduce the effort required to delete the data from each apparatus when the image data captured up to that point cannot be captured at the composition, color, or the like desired by the doctor.

In step S413, the CPU 301 of the imaging system control apparatus 106 stores the series of image data captured up to that point, and the corresponding metadata, in the image management apparatus 105. The image management apparatus 105 may include a storage apparatus, such as an HDD, a file server, or the like, or may be connected to a storage apparatus in the form of a cloud system. The image management apparatus 105 may organize and classify the locations where the image data is stored in accordance with the details of the metadata, assign tags for searching to the image data, and so on.

Note that the CPU 301 of the imaging system control apparatus 106 may cause a user interface for selecting the images to be stored (a second user interface) to be displayed in the electronic medical record terminal 104 by transmitting the series of image data captured up to that point to the electronic medical record terminal 104. Alternatively, the CPU 301 of the imaging system control apparatus 106 may display the user interface for selecting the images to be stored (the second user interface) in the display unit 304. Then, in the user interface displayed in the electronic medical record terminal 104 or the display unit 304, the doctor confirms the image data and selects the image data to be stored. In this case, in step S413, the CPU 301 of the imaging system control apparatus 106 stores the selected image data and the corresponding metadata in the image management apparatus 105. Using such a configuration makes it possible to select and store only data that is significant to the doctor from the plurality of pieces of image data, which makes it possible to shorten the time required to search for image data, save storage space, and the like.

Appropriately collecting and storing the image data, metadata, and the like as described above makes it possible to eliminate the work involved in connecting the image capturing apparatus 101 to the image management apparatus 105 or retrieving the media drive 206 from the image capturing apparatus 101 to collect and organize the image data. Additionally, based on the medical record information (the metadata), the images can be reconfirmed using various search conditions, used for conferences, and so on at a later date, and thus the doctor can view the desired image data without any hassle.

Flow of Imaging Processing in Imaging System that May Include Plurality of Image Capturing Apparatuses 101

In the descriptions given with reference to FIG. 4, a single image capturing apparatus 101 and a single electronic medical record terminal 104 are included in the imaging system. However, depending on the equipment, scale, types of medical procedures, and so on of the hospital, an image capturing system including a plurality of image capturing apparatuses 101 and electronic medical record terminals 104 may be used. The manner in which medical record information obtained from a specific electronic medical record terminal 104 is transmitted to a specific image capturing apparatus 101 in such a case will be described hereinafter.

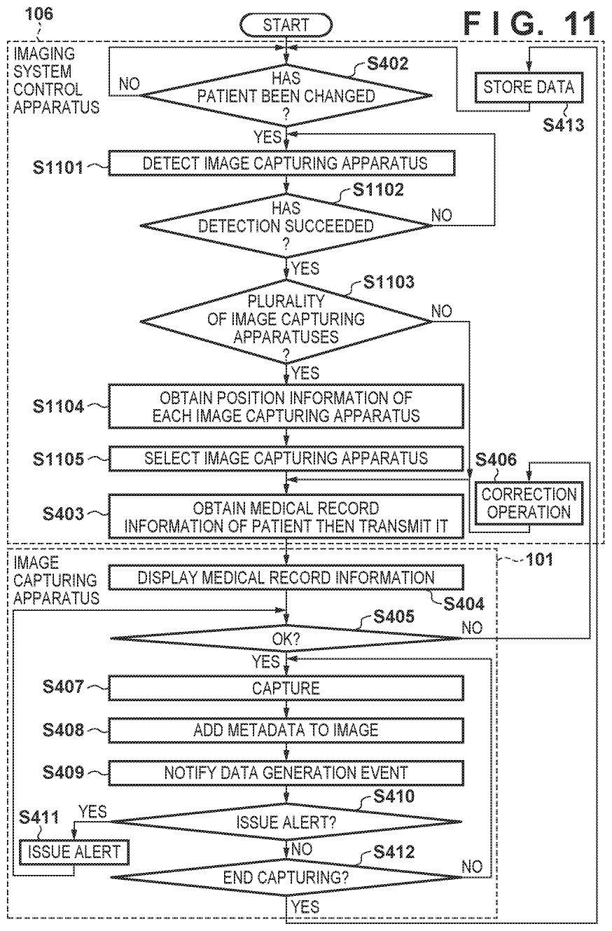

FIG. 11 is a flowchart illustrating imaging processing in an imaging system that may include a plurality of image capturing apparatuses 101. The imaging system used here may include a plurality of electronic medical record terminals 104, or may include only one electronic medical record terminal 104. When the imaging system includes a plurality of electronic medical record terminals 104, the imaging processing illustrated in FIG. 11 is executed in parallel by each electronic medical record terminal 104.

Note that of the imaging processing illustrated in FIG. 11, descriptions of points that are the same as or similar to those in FIG. 4 will omitted as appropriate. Additionally, in FIG. 11, steps that perform processes identical or similar to those in FIG. 4 are given the same reference numerals as in FIG. 4.

Additionally, it is assumed that a doctor has logged in to each electronic medical record terminal 104 prior to the imaging processing being performed. Once the doctor has logged into the electronic medical record terminal 104, the electronic medical record terminal 104 obtains the patient information (ID, name, medical department, age, sex, medical history, lesion, and the like) and the doctor information (ID, name, affiliation information such as a medical department, and the like) from the electronic medical record management apparatus 103. The electronic medical record terminal 104 can then display an electronic medical record based on the information obtained from the electronic medical record management apparatus 103.

In S1101, the CPU 301 of the imaging system control apparatus 106 attempts to detect the image capturing apparatus 101 connected to the imaging system. The detection method may be a method such as transmitting a packet to a specific IP address on the network and detecting a response thereto, or a method, such as Universal Plug and Play (UPnP), that automatically discovers and connects to devices on the network, but it goes without saying that any protocol is acceptable as long as it enables such detection.

In step S1102, the CPU 301 determines whether the image capturing apparatus 101 connected to the imaging system has been detected. If at least one image capturing apparatus 101 has been detected, the sequence moves to step S1103. However, if none have been detected, there is a possibility that the image capturing apparatus 101 is not turned on, that there is a problem with the imaging system or the network, or the like. Accordingly, the CPU 301 may notify the electronic medical record terminal 104 by displaying a notification thereof, or may notify a person in charge of maintaining the imaging system, a business operator, or the like.

In step S1103, the CPU 301 determines whether a plurality of image capturing apparatuses 101 have been detected. If a plurality of image capturing apparatuses 101 have been detected, the sequence moves to step S1104. However, if only a single image capturing apparatus 101 has been detected, the sequence moves to step S403.

In step S1104, the CPU 301 obtains position information of each of the plurality of image capturing apparatuses 101. Global Positioning System (GPS) or the like may be used for the obtainment method, or, if a plurality of Wi-Fi access points or the like are present, Wi-Fi positioning may be used. Alternatively, Radio Frequency Identifier (RFID) positioning, beacon positioning using the emission of Bluetooth signals, or positioning using broadband radio such as Ultra-Wide Band (UWB) may be used. Any method can be used as long as a positioning function provided in the image capturing apparatus 101 and the medical facility is used. Once the position information of each image capturing apparatus 101 is obtained, the sequence moves to step S1105.

In step S1105, the CPU 301 selects the image capturing apparatus 101 to be used (the image capturing apparatus 101 to which the medical record information is to be transmitted), using the position information of each of the plurality of image capturing apparatuses 101 and the position information of the electronic medical record terminal 104. Although the selection method is not particularly limited, for example, the CPU 301 can select the image capturing apparatus 101 having the shortest movement distance from the electronic medical record terminal 104. Note that the method for obtaining the location information of the electronic medical record terminal 104 is not particularly limited. For example, when the electronic medical record terminal 104 is a personal computer stationed in a specific room, the position where the electronic medical record terminal 104 is installed may be stored in the imaging system control apparatus 106 in advance. When the electronic medical record terminal 104 is in a portable form, a configuration may be employed in which the position information is obtained using a GPS or the like in the same manner as the position information of the image capturing apparatus 101. The imaging system control apparatus 106 may also be provided with information such as a floor map or the like for assisting in calculating the movement distance. Meanwhile, when a plurality of image capturing apparatuses 101 have been detected at an equal distance due to a position information obtainment error or by coincidence, the imaging system control apparatus 106 may transmit the same information to all of those image capturing apparatuses 101. Alternatively, a configuration may be employed in which the doctor can select the image capturing apparatus 101 through a user interface displayed in the electronic medical record terminal 104 or the display unit 204 of the image capturing apparatus 101. When a specific image capturing apparatus 101 is selected, the sequence moves to step S403. The processing of step S403 is the same as the processing described with reference to FIG. 4, but the destination to which the medical record information is transmitted is the image capturing apparatus 101 selected in step S1105.

Note that the configuration may be such that whether the imaging system control apparatus 106 performs the imaging processing illustrated in FIG. 4 or the imaging processing illustrated in FIG. 11 can be switched by changing the program, settings, or the like of the imaging system control apparatus 106. Additionally, a configuration may be employed in which the imaging processing illustrated in FIG. 4 and the imaging processing illustrated in FIG. 11 are switched dynamically according to the number of image capturing apparatuses 101, electronic medical record terminals 104, or the like when the imaging system control apparatus 106 is started up.

### Summary of First Embodiment

As described above, according to the imaging system (information system) of the first embodiment, in response to the patient for whom the electronic medical record is to be displayed being changed in the electronic medical record terminal 104, the imaging system control apparatus 106 obtains information (e.g., medical record information) pertaining to the post-change patient for display (the first patient) from the electronic medical record terminal 104. Then, the image capturing apparatus 101 associates the information pertaining to the first patient with an image (the first image) captured between when the patient for display is changed to the first patient and when the patient is changed to another patient. This reduces the workload of associating information pertaining to a patient with an image obtained by capturing the patient. Note that as described earlier, the imaging system control apparatus 106 may associate the information pertaining to the first patient with the first image.

Additionally, according to the imaging system of the present embodiment, the image capturing apparatus 101 can display the medical record information and the like in response to the patient for whom the electronic medical record is to be displayed being changed, and thus the user can capture an image while confirming whether the patient being captured and the medical record information associated with the image correspond correctly. Accordingly, if the medical record information is incorrect, the user can notice the medical record information being incorrect before capturing an image, and thus the image and the medical record information can be associated more accurately.

Additionally, according to the imaging system of the present embodiment, the imaging system control apparatus 106 can download image data associated with metadata (the medical record information) and store that image data in the image management apparatus 105. Accordingly, the user can confirm which patient the image data corresponds to at a later date, which makes it possible to better use the image data during medical examinations.

Additionally, according to the imaging system of the present embodiment, the image management apparatus 105 can store images in the image management apparatus 105 using classification information such as pathology, sex, patient, age, doctor, and the like based on the metadata. This makes it possible to reduce the burden of organizing the image data according to specific interest information, as well as the burden of searching for image data when retrieving image data according to specific interest information at a later date.

Additionally, according to the imaging system of the present embodiment, the imaging system control apparatus 106 can automatically download image data captured by the image capturing apparatus 101 in response to the patient for whom the electronic medical record is to be displayed being changed from the current patient to another patient in the electronic medical record terminal 104. This makes it possible, for example, to reduce the burden of importing image data from storage media. In addition, stress on doctors and patients can be suppressed even in communication infrastructures with limited network bandwidth.

Additionally, according to the imaging system of the present embodiment, the image data captured by the image capturing apparatus 101 can be automatically downloaded by the imaging system control apparatus 106 for each image captured by the image capturing apparatus 101. This makes it possible, for example, to reduce the burden of importing image data from storage media.

Additionally, according to the imaging system of the present embodiment, the user can select an image to be stored through the user interface of the imaging system control apparatus 106. This makes it possible to select and store only images, among the captured images, which are useful for medical procedures.

Additionally, according to the imaging system of the present embodiment, the user is notified when images are captured using the same medical record information continuously for at least a predetermined length of time. This makes it possible to suppress situations where another patient's medical record information is erroneously associated with an image when forgetting to switch the patient for display, when a system failure occurs, or the like.

Additionally, according to the imaging system of the present embodiment, the user is notified when a predetermined number of images have been captured using the same medical record information. This makes it possible to suppress situations where another patient's medical record information is erroneously associated with an image when forgetting to switch the patient for display, when a system failure occurs, or the like.

Additionally, according to the imaging system of the present embodiment, even if a plurality of image capturing apparatuses, electronic medical record terminals, or the like are present, the respective positional relationships thereof can be obtained, and the medical record information can be transmitted to the image capturing apparatus having the lowest movement distance for the user. This makes it possible to reduce the burden for the user to select a desired image capturing apparatus from a list of a plurality of image capturing apparatuses in a facility or the like.

Note that the hardware configuration of the image capturing apparatus 101 in the present embodiment may be the same as that of a normal digital camera, or the apparatus may be a small, portable digital camera. The various functions of the present embodiment can be realized by installing a special control program for the imaging system of the present embodiment as a control program of the image capturing apparatus 101, and then having the CPU 201 execute the control program. The hardware configuration of the imaging system control apparatus 106 may also be the same as that of a normal computer. The various functions of the present embodiment can be realized by installing a special control program for the imaging system of the present embodiment as a control program of the imaging system control apparatus 106, and then having the CPU 301 execute the control program. Accordingly, if a new control program for a business system that is not limited to medical care is prepared, an imaging system for a business purpose different from medical care can be implemented by using existing normal digital cameras and computers. An embodiment for a case where such a control program for a business system is used will be described as a second embodiment.

### Second Embodiment

The present embodiment will describe an imaging system that supports and records problem analyses on-site in a logistics support system in which a large amount of sorting is performed, such as a logistics warehouse or a factory. It is assumed that an apparatus block name, alarm position, sensor number, workpiece name, and the like are registered in advance in a PC, an in-factory server, a cloud server, or the like in a data system of a transport line (described later), for part of a transport apparatus or a transported item (a workpiece) serving as a subject in a logistics system in the present embodiment.

Although the following describes capturing images using a digital camera that can capture visible light when capturing images, it goes without saying that, as in the first embodiment, the same can be applied when the information that can be captured, the format of the camera, and the like differ according to the purpose of recording, the purpose of analysis, and the like.

The photographer will be described here as being an operator (operation/maintenance personnel) of the apparatus. Schedules, shifts, and the like are registered in the transport system, and thus the information of the photographer (a personal ID, affiliation, and the like) is also registered in the PC, server, or cloud server of the transport system, and can be accessed as necessary.

In addition, in the imaging system of the present embodiment, the configurations, connection conditions, and the like of the image capturing apparatus 101, the network 102, the setting values for image capturing conditions in the image capturing apparatus 101, the image management apparatus 105, the imaging system control apparatus 106, and the like are assumed to be the same as those described in the first embodiment unless specifically mentioned otherwise, and will therefore not be described here.

System Configuration

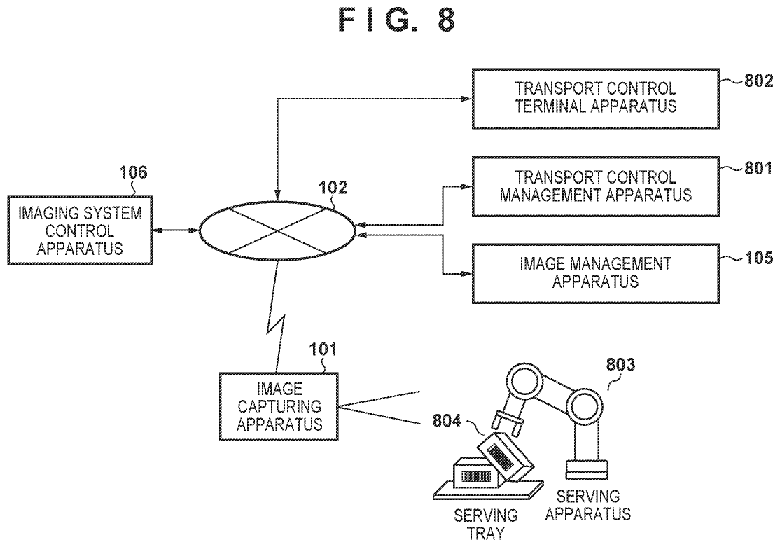

FIG. 8 is a diagram illustrating the configuration of an imaging system for a transport system. 801 indicates a transport control management apparatus. The transport control management apparatus 801 controls the transport system as a whole by controlling various transport systems, sensors, control terminals (transit sensors, presence/absence sensors, monitoring apparatuses, transport stages, and the like), various logistics apparatuses, a transport control terminal apparatus 802, and the like constituting the system as a whole, collecting and transmitting information, and the like. 803 indicates a robot-type serving apparatus. An example of a configuration in which a workpiece such as a box-shaped workpiece 804 is served is illustrated here.

The imaging system control apparatus 106 may communicate with the image capturing apparatus 101, detect image capturing, receive captured image data, and perform image processing (e.g., thumbnail extraction, data compression, and the like) according to the purpose, as described in the first embodiment. In this case, the imaging system control apparatus 106 may obtain, from the transport control management apparatus 801, the transmitted results, the reception of necessary images (downloaded from the image capturing apparatus 101, the image management apparatus 105, or the like, parameters necessary for capturing images, and the like. It is also assumed that the imaging system control apparatus 106 can obtain the detailed states, operating states, various types of information, and the like of the transport control terminal apparatus 802 and the serving apparatus 803 over the network 102. Additionally, the transport control management apparatus 801 holds the details, locations, repair results, and the like from past device problems internally, or in an external storage apparatus such as a server.

Flow of Processing for Recording Apparatus Problem

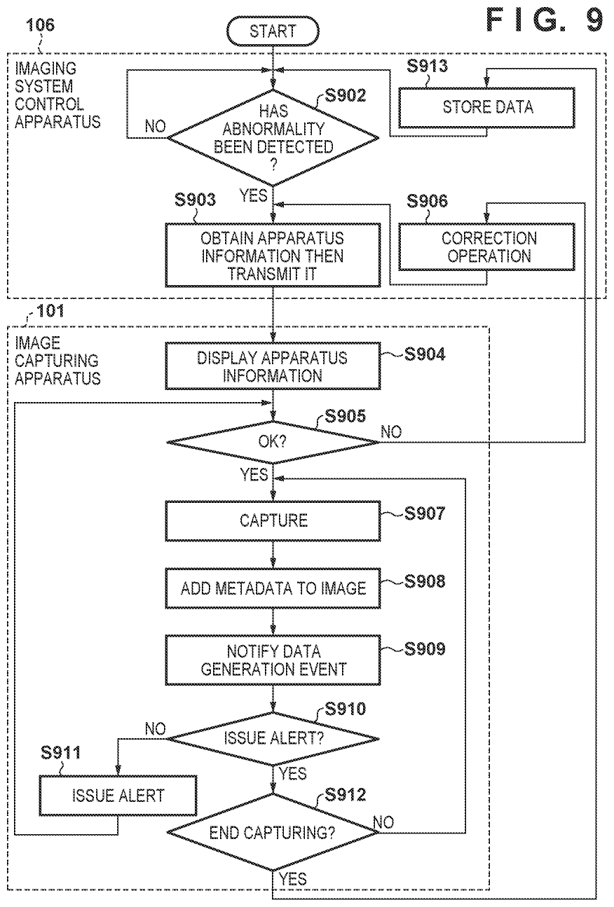

FIG. 9 is a flowchart illustrating the flow of processing for recording a problem state and the like of an apparatus using the imaging system for the transport system (recording processing) in the present embodiment. The operator is assumed to have logged in to the transport system before the recording processing is performed. The login here may be linked to an attendance management system using ID cards or the like, or the operator may log in to a personal computer or the like, and any method may be employed as long as the operator can be identified. It is also assumed that the transport control management apparatus 801 has obtained various types of apparatus information of the transport system.

The recording processing includes processing performed by the image capturing apparatus 101 and processing performed by the imaging system control apparatus 106. Unless otherwise specified, the processing by the image capturing apparatus 101 is realized by the CPU 201 of the image capturing apparatus 101 executing a control program. Additionally, unless otherwise specified, the processing by the imaging system control apparatus 106 is realized by the CPU 301 of the imaging system control apparatus 106 executing a control program.

Like the imaging processing of the first embodiment (FIGS. 4 and 11), the division of processing among the apparatuses is not limited to the following descriptions and the division indicated in FIG. 9.

In step S902, the CPU 301 of the imaging system control apparatus 106 detects whether problem information has been generated in a specific apparatus. If so, the sequence moves to step S903. If not, the processing stands by at step S902.

In S903, the CPU 301 obtains information on the apparatus in which the anomaly has occurred, and transmits the information to the image capturing apparatus 101. The information obtained here, the information transmitted to the image capturing apparatus 101, and so on can be various types of information, such as an apparatus ID, position information, details of the problem, the image capturing date, a history of problems, a history of images captured, and the like. It is assumed here that the CPU 301 transmits, to the image capturing apparatus 101, mainly auxiliary information for the operator to capture an image of the apparatus. For example, the CPU 301 transmits the apparatus ID, the position information, the problem details, and the time to the image capturing apparatus 101 as metadata.

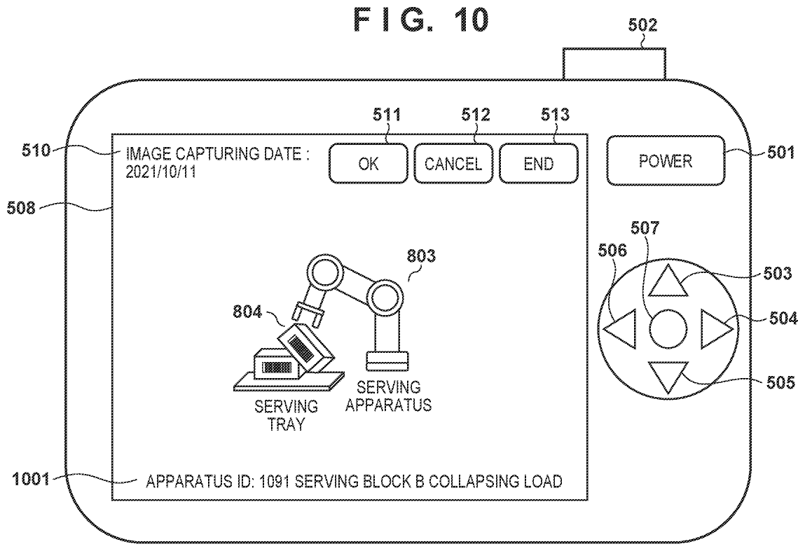

In step S904, the CPU 201 of the image capturing apparatus 101 displays the metadata transmitted from the imaging system control apparatus 106 in the display 508 illustrated in FIG. 10, as indicated by apparatus information 1001. This makes it easy for the operator to confirm that the apparatus to be captured matches. The operator confirms the displayed information and selects the OK button 511 if the apparatus information (including the problem information obtained from the apparatus) is correct. If the actual apparatus information is wrong due to some error (for example, when detection by a sensor in the system has malfunctioned), the operator presses the cancel button 512.

In step S905, the CPU 201 of the image capturing apparatus 101 determines whether the OK button 511 has been detected as being selected. If the OK button 511 has been detected as being selected, the sequence moves to step S907. If the cancel button 512 has been detected as being selected, the sequence moves to step S906.

In step S906, the CPU 301 of the imaging system control apparatus 106 attempts to collect and update the apparatus information in which a problem has occurred again. The sequence then moves to step S903.

In step S907, the CPU 201 of the image capturing apparatus 101 captures an image and stores the image data in the RAM 203. Note that if the end button 513 or the like provided in the image capturing apparatus 101 is not pressed, the CPU 201 may treat the image data as having been stored temporarily, and discard the image data.

In step S908, the CPU 201 adds the metadata transmitted in step S903 to the image data captured in step S907. Although the method for adding the metadata is not particularly limited, the CPU 201 can, for example, write the metadata to a metadata region of the image data or the like. Alternatively, the CPU 201 may control the file generation unit 212 to generate a metadata file containing the metadata, and associate the image data file with the metadata file.

In step S909, the CPU 201 notifies the imaging system control apparatus 106 of a data generation event. The “data generation event” is an event indicating that image data to which metadata has been added has been generated by the image capturing apparatus 101. Accordingly, upon receiving the notification of the data generation event, the CPU 301 of the imaging system control apparatus 106 can recognize that new image data has been generated by the image capturing apparatus 101, and can receive (download) that image data.

Note that the timing at which the CPU 301 of the imaging system control apparatus 106 downloads the image data from the image capturing apparatus 101 is not particularly limited. For example, the CPU 201 of the image capturing apparatus 101 may hold the image data in a storage region (the ROM 202, the media drive 206, or the like), and the CPU 301 of the imaging system control apparatus 106 may download the image data at a later time. Such a configuration makes it possible to alleviate stress for the photographer even in an infrastructure environment in which the download time is delayed for each instance of image capturing, such as with transport systems that capture a large number of images or when the bandwidth of the network 102 is limited.

In step S910, the CPU 201 of the image capturing apparatus 101 determines whether to alert (notify) the operator. If it is determined that the operator is to be alerted, the sequence moves to step S911, and if not, the sequence moves to step S912. For example, when the same metadata has been used for at least a predetermined length of time, or when more than a predetermined number of images have been captured, an alert may be issued inquiring as to whether an alarm needs to be canceled or the like due to communication between the transport control management apparatus 801 and the transport control terminal apparatus 802, a forgotten operation of the transport control management apparatus 801, or the like. Note that the method for making the alert is similar to that illustrated in FIG. 6 and will therefore not be described here.

In step S911, the CPU 201 alerts the user to the possibility of the transport control management apparatus 801, the transport control terminal apparatus 802, or the like being erroneously handled. The sequence then moves to step S905.

In step S912, the CPU 201 determines whether to end the capturing of images. When the end button 513 is pressed, the CPU 201 clears the metadata held by the image capturing apparatus 101. The sequence then moves to step S913. The method for clearing the metadata is similar to that illustrated in FIG. 7 and will therefore not be described here.

In this manner, clearing metadata such as device information and the like each time an image of a single apparatus problem is captured makes it possible to suppress erroneously associating other apparatus anomaly information with the captured image data. If the device information is not entered, image capturing may be suppressed by making it impossible to capture an image even if the release button 502 is pressed.

In step S913, the CPU 301 of the imaging system control apparatus 106 stores the series of captured data captured up to that point, and the metadata, in the image management apparatus 105. The sequence then returns to step S902, where the CPU 301 returns to a state of standing by for an anomaly to be detected.

Note that as described in the first embodiment, it goes without saying that the storage location may be arranged in accordance with the details of the metadata during storage, the searchability may be increased by adding tags, and the like. Doing so makes it possible to eliminate the burden on the operator for making an operational connection with the image capturing apparatus 101, organizing the captured data using a media drive, and the like. Additionally, the images can be organized in accordance with problem attributes, apparatus IDs, and the frequency of occurrence, and thus the operator can store the problems, record methods for improvement, and so on, without the burden of organizing the image data.

Flow of Recording Processing in Transport System that May Include Plurality of Image Capturing Apparatuses 101

Like FIG. 1 in the first embodiment, the image capturing apparatus 101 and the transport control management apparatus 801 in FIG. 8 have been described as being in a one-to-one relationship. However, depending on the facilities, scale, and the like of the logistics warehouse or factory, the characteristics of the apparatuses, and the like, there may be a system in which there are a plurality of images capturing apparatuses 101, transport control management apparatuses 801, or the like. The manner in which metadata such as the apparatus ID, position information, problem details, and time obtained from the transport control management apparatus 801 is transmitted to a specific image capturing apparatus in such a case will also be described.

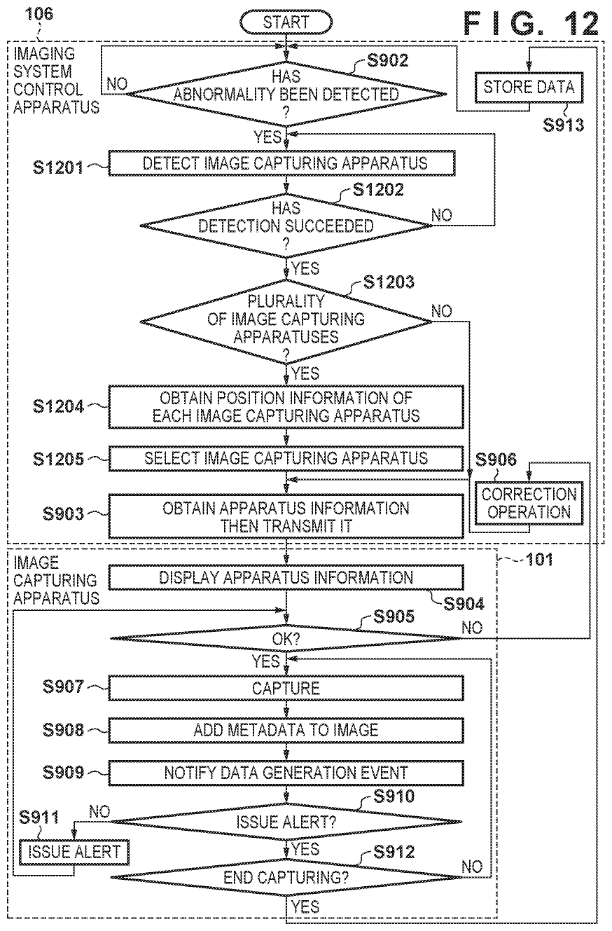

FIG. 12 is a flowchart illustrating recording processing in a transport system that may include a plurality of image capturing apparatuses 101. The transport system used here may include a plurality of transport control management apparatuses 801, or may include only one transport control management apparatus 801. When the transport system includes a plurality of transport control management apparatuses 801, the recording processing illustrated in FIG. 12 is executed in parallel for each transport control management apparatus 801.

Note that of the recording processing illustrated in FIG. 12, descriptions of points that are the same as or similar to those in FIG. 9 will omitted as appropriate. Additionally, in FIG. 12, steps that perform processes identical or similar to those in FIG. 9 are given the same reference numerals as in FIG. 9.

Additionally, the fact that the operator is logged in to the transport system prior to the execution of the recording processing is the same as in FIG. 9.

In step S902, the CPU 301 of the imaging system control apparatus 106 determines whether an anomaly in the logistics system has been newly detected. If an anomaly has been detected, the CPU 301 determines that capturing of the new apparatus anomaly is to start. The sequence then moves to step S1201. If an anomaly has not been detected, the sequence returns to step S902, and the CPU 301 performs the detection processing again.

In step S1201, the CPU 301 attempts to detect the image capturing apparatuses connected to the system. The detection method may be a method such as transmitting a packet to a specific IP address on the network and detecting a response thereto, or a method, such as Universal Plug and Play (UPnP), that automatically discovers and connects to devices on the network, but it goes without saying that any protocol is acceptable as long as it enables such detection.

In step S1202, the CPU 301 determines whether an image capturing apparatus connected to the system has been detected. If at least one image capturing apparatus has been detected, the sequence moves to step S1203. However, if none have been detected, there is a possibility that the image capturing apparatus is not turned on, that there is a problem with the system or the network, or the like. Accordingly, the CPU 301 may make a notification by displaying an indication that a malfunction may have occurred, or may notify the person in charge of maintaining the system, the business operator, or the like.

In step S1203, the CPU 301 determines whether a plurality of image capturing apparatuses have been detected in step S1202. If a plurality of image capturing apparatuses have been detected, the sequence moves to step S1204. However, if only a single image capturing apparatus has been detected, the sequence moves to step S903.

In step S1204, the CPU 301 obtains position information of each image capturing apparatus. Global Positioning System (GPS) or the like may be used for the obtainment method, or, if a plurality of Wi-Fi access points or the like are present, Wi-Fi positioning may be used. It goes without saying that any method using a positioning unit provided in an image capturing apparatus and a facility, such as positioning methods using Radio Frequency Identifier (RFID) positioning, beacon positioning using the emission of Bluetooth signals, or positioning using broadband radio such as Ultra-Wide Band (UWB), may be used. Once the position information of each image capturing apparatus has been obtained, the sequence moves to step S1205.

In step S1205, using the position information of each of the plurality of image capturing apparatuses 101 and the position information of the transport control management apparatus 801, the CPU 301 determines the image capturing apparatus 101 having the shortest movement distance as the target for transmitting the apparatus information. Note that the method for obtaining the position information of the transport control management apparatus 801 is not particularly limited. For example, when the transport control management apparatus 801 is a personal computer stationed in a specific room, the position where the transport control management apparatus 801 is installed may be stored in the imaging system control apparatus 106 in advance. When the transport control management apparatus 801 is in a portable form, a configuration may be employed in which the position information is obtained using a GPS or the like in the same manner as the position information of the image capturing apparatus 101. The imaging system control apparatus 106 may also be provided with information such as a floor map or the like for assisting in calculating the movement distance. Meanwhile, when a plurality of image capturing apparatuses 101 have been detected at an equal distance due to a position information obtainment error or by coincidence, the imaging system control apparatus 106 may transmit the same information to all of those image capturing apparatuses 101. Alternatively, a configuration may be employed in which the operator can select the image capturing apparatus 101 through a user interface displayed in the transport control management apparatus 801 or the display unit 204 of the image capturing apparatus 101. When a specific image capturing apparatus 101 is selected, the sequence moves to step S903. The processing of step S903 is the same as the processing described with reference to FIG. 9, but the destination of the metadata is the image capturing apparatus 101 selected in step S1205.

Note that the configuration may be such that whether the imaging system control apparatus 106 performs the recording processing illustrated in FIG. 9 or the recording processing illustrated in FIG. 12 can be switched by changing the program, settings, or the like of the imaging system control apparatus 106. Additionally, a configuration may be employed in which the recording processing illustrated in FIG. 9 and the recording processing illustrated in FIG. 12 are switched dynamically according to the number of image capturing apparatuses 101, transport control management apparatuses 801, or the like when the imaging system control apparatus 106 is started up.

### Summary of Second Embodiment

As described thus far, according to the imaging system of the second embodiment, the image capturing apparatus 101 can automatically obtain apparatus information, operator information, and the like in response to the state of the transport apparatus (e.g., anomaly detection) changing. Accordingly, the image data can easily be associated with metadata such as the apparatus information, the operator, and the like. This makes it possible to reduce the burden on the operator for associating or entering information, which can be expected to encourage the use of the image data.

Additionally, according to the imaging system of the present embodiment, the image capturing apparatus 101 can display apparatus information, operator information, and the like in response to the state of the transport apparatus (e.g., anomaly detection) changing. Accordingly, the operator can capture images while confirming that the apparatus, the image data, and the metadata correspond correctly. When the apparatus information or the like is incorrect, the operator can notice the problem before capturing an image, and thus the image data and the metadata can be more accurately associated with each other.

Additionally, according to the imaging system of the present embodiment, the image capturing apparatus 101 can display apparatus information, operator information, and the like in response to the state of the transport apparatus (e.g., anomaly detection) changing. Accordingly, the operator can confirm whether the apparatus, the image data, and the associated metadata correspond correctly. If the correspondence is incorrect, the operator can correct or update the apparatus information. Accordingly, the metadata associated with the image data can be corrected by the operator when the apparatus information or the like is incorrect.

Additionally, according to the imaging system of the present embodiment, the image data and the metadata can be downloaded and stored in a storage apparatus or the like. Which apparatus, operator, or the like captured the image data can therefore be confirmed at a later date, which makes it possible to make better use of the image data during maintenance, inspections, or repairs.

Additionally, according to the imaging system of the present embodiment, image data and metadata can be associated with each other and downloaded, and then stored in a storage apparatus or the like using classification information such as an apparatus ID, anomaly details, a date/time, a frequency, a location, and so on, based on the supplementary information in the metadata. This makes it possible to reduce the burden of organizing the image data according to specific interest information, as well as the burden of searching for image data when retrieving image data according to specific interest information at a later date.

Additionally, according to the imaging system of the present embodiment, image data and metadata can be associated and automatically downloaded at the timing at which the state of the transport apparatus (e.g., anomaly detection) switches. This makes it possible, for example, to reduce the burden of importing image data from storage media. In addition, stress on operators, apparatuses, and the like can be suppressed even when the image data communication infrastructure provides limited network bandwidth.

Additionally, according to the imaging system of the present embodiment, the image data and metadata can be associated and automatically downloaded for each instance of image capturing by the image capturing apparatus. This makes it possible, for example, to reduce the burden of importing image data from storage media.

Additionally, according to the imaging system of the present embodiment, images downloaded having associated the apparatus information with the image data can be confirmed by the transport control system, and the image data to be stored can be selected and then transmitted to the storage apparatus. This makes it possible to select and store only images, among the captured images, which are useful for maintenance, inspections, or repairs.

Additionally, according to the imaging system of the present embodiment, a notification can be made for confirming the apparatus information when a certain amount of time has passed after specific apparatus information has been transmitted to the image capturing apparatus. This makes it possible to suppress situations where the switching of the state of the transport apparatus (e.g., anomaly detection) is missed, or, when a system failure occurs, incorrect apparatus information is associated with the image data or image data with which incorrect apparatus information has been associated is stored.

Additionally, according to the imaging system of the present embodiment, a notification can be made for confirming the apparatus information when a certain number of images have been captured after specific apparatus information has been transmitted to the image capturing apparatus. This makes it possible to suppress situations where the switching of the state of the transport apparatus (e.g., anomaly detection) is missed, or, when a system failure occurs, incorrect apparatus information is associated with the image data or image data with which incorrect apparatus information has been associated is stored.

Additionally, according to the imaging system of the present embodiment, even when a plurality of image capturing apparatuses, transport control management apparatuses, or the like are present, the respective positional relationships can be obtained, the image capturing apparatus having the shortest movement distance for the operator can be selected as the destination for transmitting the apparatus information, and the apparatus information can be transmitted. This makes it possible to reduce the burden for the photographer to select a desired image capturing apparatus from a list of a plurality of image capturing apparatuses in a facility or the like.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2022-099802, filed Jun. 21, 2022 which is hereby incorporated by reference herein in its entirety.

What is claimed is:

1. An information system comprising:
a control apparatus including a first obtainment unit configured to obtain first information pertaining to a first patient from an electronic medical record terminal in response to a patient for display of an electronic medical record changing to the first patient in the electronic medical record terminal, the control apparatus being capable of communicating with the electronic medical record terminal; and
an association unit configured to associate the first information with a first image captured between when the patient for display is changed to the first patient and when the patient for display is changed to another patient, the first image being captured by a specific image capturing apparatus capable of communicating with the control apparatus.

2. The information system according to claim 1,
wherein the control apparatus includes a transmission unit configured to transmit the first information to the specific image capturing apparatus in response to the first information being obtained,
the specific image capturing apparatus is included in the information system, and includes a reception unit configured to receive the first information from the control apparatus, and
the association unit is included in the specific image capturing apparatus, and associates the first information received from the control apparatus with the first image.

3. The information system according to claim 2,
wherein the specific image capturing apparatus includes a first display unit configured to display at least some of the first information in response to the first information being received from the control apparatus.

4. The information system according to claim 2,
wherein the control apparatus includes a second obtainment unit configured to obtain the first image with which the first information is associated from the specific image capturing apparatus in response to the patient for display changing from the first patient to another patient.

5. The information system according to claim 2,
wherein the control apparatus includes a second obtainment unit configured to obtain the first image with which the first information is associated from the specific image capturing apparatus in response to the first information being associated with the first image.

6. The information system according to claim 4,
wherein the specific image capturing apparatus includes:
a second display unit configured to display a first user interface including the first image and a cancellation option in response to the first image being captured; and
a notification unit configured to make a first notification to the control apparatus in response to a first time passing without the cancellation option being selected in the first user interface, and
the second obtainment unit obtains the first image with which the first information is associated from the specific image capturing apparatus when the first notification is made.

7. The information system according to claim 4,
wherein the control apparatus includes a storage unit configured to store the first image obtained from the specific image capturing apparatus in a storage apparatus.

8. The information system according to claim 7,
wherein the control apparatus includes a third display unit configured to display a second user interface for selecting an image to be stored, and
the storage unit stores the first image in the storage apparatus when the first image is selected in the second user interface.

9. The information system according to claim 1, further comprising:
a second notification unit configured to make a second notification to a user in response to a second time passing after the patient for display has changed to the first patient and before the patient for display changes to another patient.

10. The information system according to claim 1, further comprising:
a third notification unit configured to make a third notification to a user in response to a predetermined number of images being captured by the specific image capturing apparatus after the patient for display has changed to the first patient and before the patient for display changes to another patient.

11. The information system according to claim 1,
wherein the control apparatus includes a selection unit configured to, when a plurality of image capturing apparatuses are capable of communicating with the control apparatus, select an image capturing apparatus, among the plurality of image capturing apparatuses, to use as the specific image capturing apparatus, based on a position of the electronic medical record terminal and a position of each of the plurality of image capturing apparatuses.

12. An information processing method comprising:
obtaining first information pertaining to a first patient from an electronic medical record terminal by a first obtainment unit of a control apparatus capable of communicating with the electronic medical record terminal, the obtaining being performed in response to a patient for display of an electronic medical record changing to the first patient in the electronic medical record terminal; and
associating, by an association unit, the first information with a first image captured between when the patient for display is changed to the first patient and when the patient for display is changed to another patient, the first image being captured by a specific image capturing apparatus capable of communicating with the control apparatus.

13. A non-transitory computer-readable storage medium which stores a program for causing a computer system, which includes a control apparatus capable of communicating with an electronic medical record terminal, to execute an information processing method comprising:
obtaining first information pertaining to a first patient from the electronic medical record terminal, the obtaining being performed in response to a patient for display of an electronic medical record changing to the first patient in the electronic medical record terminal; and
associating the first information with a first image captured between when the patient for display is changed to the first patient and when the patient for display is changed to another patient, the first image being captured by a specific image capturing apparatus capable of communicating with the control apparatus.
